(19) United States
(12) Patent Application Publication
(10) Pub. No.: US 2014/0190482 A1
(43) Pub. Date: Jul. 10, 2014

(54) Controller device, system and method for improved patient respitory care

(71) Applicant: Reuben Eugene Wade, Hempstead, TX (US)

(72) Inventor: Reuben Eugene Wade, Hempstead, TX (US)

(21) Appl. No.: 13/646,626

(22) Filed: Jan. 9, 2013

(51) Int. Cl. A61M 16/20; A61M 16/12; A61M 16/04; A61M 16/06; A61M 16/08; A61M 16/16; A61M 16/10

(52) U.S. Cl. CPC A61M 16/201; A61M 16/16; A61M 16/122; A61M 16/1005; A61M 16/06; A61M 16/0875; A61M 16/047; A61M 16/0808
USPC 128/203.25; 128/203.12

(57) ABSTRACT

This invention provides an improved respiratory care system for patients. This system uses vapor generated by a humidifier device to provide a humidified vapor mist directly to a trachea patient via a tubing element. A vapor controller attaches to a conventional humidifier device and controls the flow rate of vapor mist from the humidifier to the patient. A tubing element attaches to the outlet of the vapor controller and channels to humidified vapor directly to the patient. In one embodiment of the present invention, the tubing element can attach directly to the trachea of the patient through a trachea mask.

### FIELD OF THE INVENTION

This invention relates to a method and system for respitory care for patients and in particular this invention relates to a controller device and method and system for improved respitory care of patients. Still, more specifically, this invention relates to a humidity controller system that enhances the effectiveness humidified vapor in the treatment of respitory patients.

### BACKGROUND OF THE INVENTION

A humidifier is a household appliance that increases humidity (moisture) in the air. The air can be in a single room or in the entire house. Hospitals also use humidifiers for their patients. In hospitals, patient humidification is a method of artificially conditioning respiratory vapors for the patient during therapy. This process involves generating a humidified vapor being delivered to a patient. These vapors can be warmed and in instances filtration of the vapor can occur. If these three measures are not performed to compensate for the natural conditioning of air by the respiratory system, pulmonary infections and lung tissue damage may occur.

In hospitals, many patients have tracheostomy procedures. A tracheotomy is a surgical procedure in which a cut or opening is made in the windpipe, or trachea, and a tube is inserted into the opening to bypass an obstruction. This tube is called a tracheostomy tube or trach tube. This tracheotomy procedure is performed if enough air is not getting to the lungs and if the person cannot breathe without help. In this procedure, a tube is usually placed through this opening to provide an airway and to remove secretions from the lungs. A trachea is also inserted into the patient if the patient is having problems with mucus and other secretions getting into the windpipe because of difficulty swallowing. Several reasons account for why air cannot get into a patient's lungs. First, the windpipe may be blocked by a swelling; by a severe injury to the neck, nose or mouth, by a large foreign object; by paralysis of the throat muscles; or by a tumor. Additionally, the patient may be in a coma or need a ventilator to pump air into the lungs for a long period of time. Tracheotomies are also commonly performed on infants who are born prematurely, since their lungs may not be fully developed at the time of birth.

In the human body, the nose and mouth provide warmth, moisture and filtration for the air a person breathes. Having a tracheostomy tube, however, by-passes these mechanisms so humidification must be provided to keep secretions thin and to avoid mucus plugs. In the general trach patient process, a mist collar (trach mask) with aerosol tubing over the trach with the other end of tubing is attaches to a nebulizer bottle and air compressor. Sterile water goes into the nebulizer bottle (do not overfill, note line guide). Oxygen can also be delivered via the mist collar if needed.

As a life-saving procedure, millions of tracheotomies are performed every year in the United States and all over the world. Since tracheas remain in place, in most cases, for an indeterminate amount of time, it is not uncommon for the throat and respirator area to become dried out, which can cause discomfort and possibly exacerbation of the condition for the patient. To help combat this, medical facilities typically attach a wall-mounted humidifier to the trachea tubing. However, this can prove challenging for the patient, as he/she is remanded to the bed while moisturizing the throat, unable to move until the procedure is complete. Additionally, when home, patients must use extremely cumbersome, and quite costly, humidifier devices in order to fight the trachea's dryness.

Conventional hospital treatments, for respiratory patients that have a trachea incorporate an air compressor and an air ventilator. The air compressor supplies air for the trachea patient. However, compressed air is warmer than desired for the trachea patient and would cause dryness in the trachea. The ventilator supplies cool air that mixes with the compressed air such that the air received at the patient's trachea is cooler and reduces dryness in the trachea. In to the functioning of the air compressor and the ventilator, in the conventional hospital treatments for respiratory patients, a nebulizer combines the mixed air from the compressor and ventilator with water. These components, the air compressor, ventilator and nebulizer form the conventional system used in hospitals to provide air to the patient during respiratory treatments.

Although this system is sufficient for treating patients in the hospital environment, once the hospital discharges the patient, the patient must still continue the respiratory treatments at home. Conventional home treatments for trachea patients comprise the use of the conventional humidifier device. In order to moisten the air, the humidifier device dispenses humidified air into the atmosphere of a room. Constant dispensing of humidified air into the atmosphere will eventually result in a humidified room that is conducive for the respiratory patient. As the patient breathes in the humidified air through the trachea, the moister in the humidified air will prevent dryness to the trachea.

This current system for home use implemented for the trachea patient is very inefficient. First, the humidity in the patient's room must reach a desired humidity level. This level is far less than the level of humidity in the air that a patient receives during hospital treatments. Second, the humidified air produced by the humidifier s directed to the room in general and not the patient in particular. In this current home system, a humidifier creates far more humidified air than the patient needs.

There remains a need for a respiratory method and system for a patient's use at home that provides air with sufficient moisture such that dryness does form in the patient's trachea. There also remains a need for a system that is efficient in the provision of humidified are to a trachea patient during respiratory treatments for that patient at that patient's home.

### SUMMARY OF THE INVENTION

The present invention provides significant benefits over conventional home and hospital treatments for respiratory patients. The present invention is a compact, portable unit that facilitates distribution of cool, refreshing mist to tender throat and respiratory areas. This invention handily alleviates the dry, irritation the throat and lungs that typically result from extended use of tracheas. In this manner, patients who must be outfitted with these medical devices can receive their needed therapy with uninterrupted comfort. Also, this device may be used with a nebulizer for breathing treatments as well as to unstop block nasal passages simply by disconnecting the trachea mask and connecting to an aerosol mask.

This invention provides an improved respiratory care system for patients. This system uses vapor generated by a humidifier device to provide humidified air directly to a trachea patient via a tubing element. A vapor controller attaches to a conventional humidifier device and controls the flow rate of vapor from the humidifier to the patient. A tubing element attaches to the outlet of the vapor controller channels to humidified vapor directly to the patient. In one embodiment of the present invention, the tubing element will attach directly to the trachea of the patient through a trachea mask.

The device and system of the present invention not only assists patients with a trachea, but also with some other respiratory conditions. As a result, this invention is multifunctional and helps promote overall health and well-being with simple application. In addition to use in the home, this invention is adaptable for application in health care facilities such as hospitals and rehabilitation centers.

In another embodiment of the present invention, the vapor controller can facilitate the implantation of other respiratory activities such as breathing treatments. In addition, the vapor controller has an input port for the intake of oxygen. The vapor controller can facilitate the combining of the oxygen with the humidified vapor and channel the combined vapor to the patient. For some breathing treatments, an aerosol mask fit over the patient's nose and/or mouth and attaches to the tubing element.

In another embodiment of the present invention, a nebulizer device can be attached to the tubing element to facilitate a breathing treatment for the patient. These treatments can include bronchitis. The vapor controller is adaptable to function independent of the humidifier. In addition, the vapor controller is attachable to humidifiers of various sizes.

In another embodiment of the present invention, a strainer is attached to the bottom chamber of the vapor controller to allow a patient to insert a vapor pad into the strainer to for a vaporizing mist that will unstop blocked nasal passage.

### DESCRIPTION OF THE DRAWINGS

FIG. 1 is an illustration of the vapor controller system of the present invention designed to be attached to a standard humidifier device.

FIG. 2 is an illustration of the system of the present invention incorporating the vapor controller and a standard humidifier device.

FIG. 3 is an illustration of the mini-vapor controller system of the present invention designed to be attached to a mini-humidifier device.

FIG. 4 is an illustration of the system of the present invention incorporating the mini-vapor controller and a mini-humidifier device.

FIGS. 5a and 5b show high humidity adapter plugs for the vapor controller device.

FIG. 6 shows a vent barrel positioned inside the channel of the humidity adapter in the vapor controller.

FIG. 7 shows a configuration of an oxygen diluter that can be implemented in the present invention.

FIG. 8 shows the engagement of the oxygen diluter with the vent barrel in the present invention.

FIG. 9 shows a patient connected to a vapor controller system of the present invention that is attached to a standard humidifier device that enables to the patient to receive humidified vapor mist.

FIG. 10 shows a patient connected to a vapor controller system of the present invention that is attached to a min-humidifier device that enables to the patient to receive humidified vapor mist.

FIG. 11 is a flow diagram of the steps in the implementation of the method of the present invention.

### DETAILED DESCRIPTION OF THE INVENTION

The present invention is system for improved respiratory care of a patient. The primary application of the system, controller and method of the present invention is home health care of respiratory patients. However, the present invention has applications in other health care environments such as hospitals and rehabilitation centers. The present invention incorporates as a primary component, a vapor mist controller. This vapor mist controller has a modular construction and is adaptable and reconfigurable which enables a patient to use this vapor mist controller for various respiratory applications. For example, the patient can use this vapor mist controller as a high humidity adapter to moisten the trachea, to connect with an oxygen machine, to use with a nebulizer for patient breathing treatments and to unstop a blocked nasal passage.

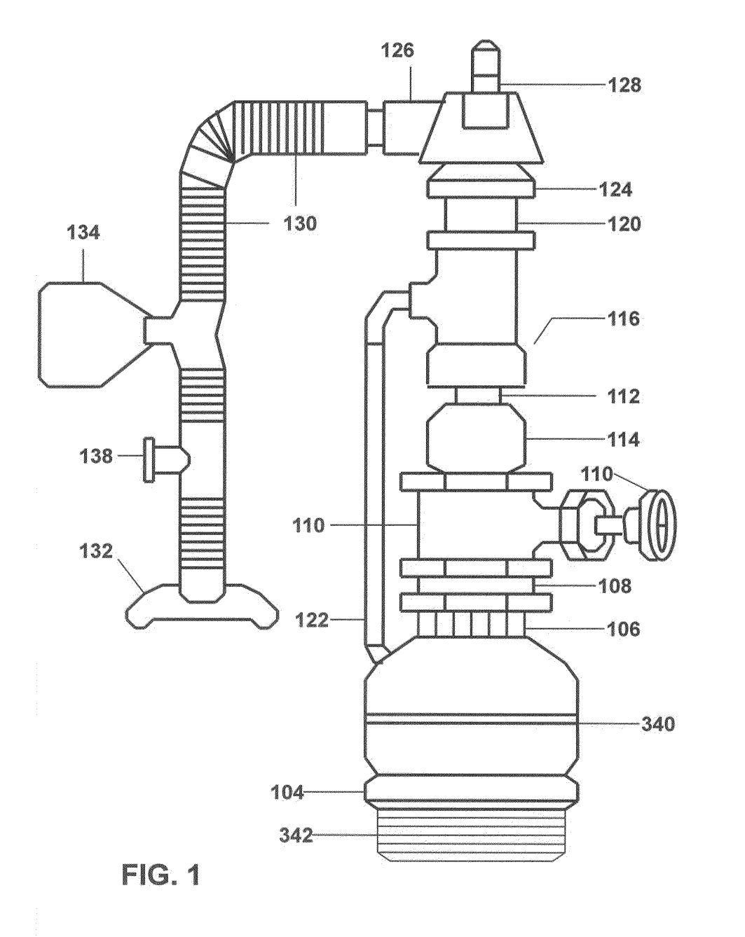

In a primary embodiment, the present invention provides an improved respiratory care system for patients. FIG. 1 is an illustration of the vapor mist controller system of the present invention designed to be attached to a standard ultrasonic humidifier device. This system comprises the vapor mist controller, a corrugated tube and a trachea mask. In the implementation of this system a humidifier device is connected to the vapor mist controller and supplies a humidified vapor mist. The vapor mist from the humidifier device travels through the vapor mist controller. The vapor mist controller attaches to a corrugated tube. The humidified vapor mist travels from the vapor mist controller and into and through the corrugated tube. At the end of the corrugated tube is a trachea mask connected to a trachea in the patient. The patient breathes in the humidified vapor mist. The moisture in the vapor mist reduces dryness in the patient's trachea.

As shown in FIG. 1, the vapor mist controller comprises a humidifier adapter 104 that facilitates the connection of the vapor mist controller to a humidifier source. Preferably, the humidifier source can be a conventional commercial ultrasonic humidifier currently used in homes. The humidifier adapter can be of various designs to engage and connect with the outlet of the humidifier. The humidifier adapter 104 can have a circular shape with inner threads to engage a strainer or the humidifier device. This humidifier outlet forms the initial opening of a channel for travel of the humidified vapor mist from the humidifier and through vapor mist controller. Attached to the humidifier adapter 104 is a male adapter 108. The bottom end of the male adapter connects to a bottle top connection 106 that is attached to the humidifier adapter 104. Inserted into the top end of the bottle top connection is the male adapter 108. This male adapter has a linear cylindrical shape with top and bottom ends and a middle section. The bottom end of this male adapter is inserted into the top end of the bottle top connection 106. The top end of the male adapter can have threads for engaging a flow control valve 110. The male adapter can comprise ¾ inch PVC material. The control valve 110 is a one-way flow control valve. The valve has a bottom end connected to the male adapter 108. As with conventional flow control valves, a restriction element inside the valve alters the size of the valve channel to enable an operator to adjust the flow of the humidified vapor through the valve. In addition, the restriction element can have the capability to restriction the flow of humidified vapor to one direction. Humidified vapor enters the valve from the male adapter 108 and flows out of the valve through the upper end. In this embodiment of the present invention, the control valve can be a ½ inch one-way PVC control valve. A PVC pipe nipple 112 connects the flow control valve to a reducing bushing 114. The nipple pipe 112 can be a ¾ inch nipple pipe. In addition, the reducing bushing can be ½ inch by ¾ inch. A pressure line adapter 116 is connected to the reducing bushing 114. This pressure line adapter 116 can comprise a plastic/nylon material. The inner diameter is approximately 22 millimeters. This pressure line adapter comprises a circular shape with top and bottom open ends forming a channel. This pressure line adapter can also have an opening for insertion of a multi-vent barrel 120. Connected to the opening in the pressure line adapter 116 is a pressure tube 122. This pressure tube also connects to the opening in the humidifier adapter 104. The pressure tube 122 forms a pressure line from 116 to 340. This pressure tube helps regulate the pressure of the humidified vapor mist traveling through the vapor mist controller 340.

The multi-vent barrel 120 can be an optional component of the vapor mist controller. The multi-vent barrel is used to control the amount of vapor mist is released from the vapor mist controller to the patient. The multi-vent barrel 120 can also regulate the amount of oxygen from the oxygen diluter that is mixed with the vapor mist. A mist retainer and adjustment control locking ring 124 fits around and between the multi-vent barrel and the oxygen diluter 128 to secure the multi-vent barrel in place. The high humidity adapter 126 can have a generally circular shape with a bottom, a top, and a side opening. The bottom opening connects to the multi-vent barrel and receives the vapor mist flowing though the multi-vent barrel. The side opening is connected to the corrugate tube 130 and channels the vapor mist into the corrugated tube. The high humidity adapter 126 can have an optional top opening to facilitate the connecting of an oxygen source for via connector 128 for oxygen dilution of the vapor mist.

One end of the corrugated tube 130 attaches to the outlet of the high humidity adapter and the other end of the corrugated tube is attached to the trachea mask 132. Attached along the corrugated tubing is a drainage bag 134. This bag collects the condensation that accumulates as the vapor mist travels through the tube to the patient. Also attached to the corrugated tube 130 is a nylon tube adapter to facilitate the connection of a Tee Adapter valve connector 138 for connection of a nebulizer that a patient uses during breathing treatments. All of the attachments to the corrugated tube, the drainage bag 134, the nylon tube adapter and the Tee Adapter valve connector 138 are optional components but all do facilitate an optimal implementation of the system of the present invention. The trachea mask 132 attaches to the patient's trachea and provides humidified vapor mist from the humidifier to maintain sufficient moister in the trachea. For non-trachea applications such as breathing treatments, an aerosol-mask replaces the trachea mask at the end of the corrugated tubing. This aerosol mask fits over the patient's nose and/or mouth can be implemented instead of the trachea mask.

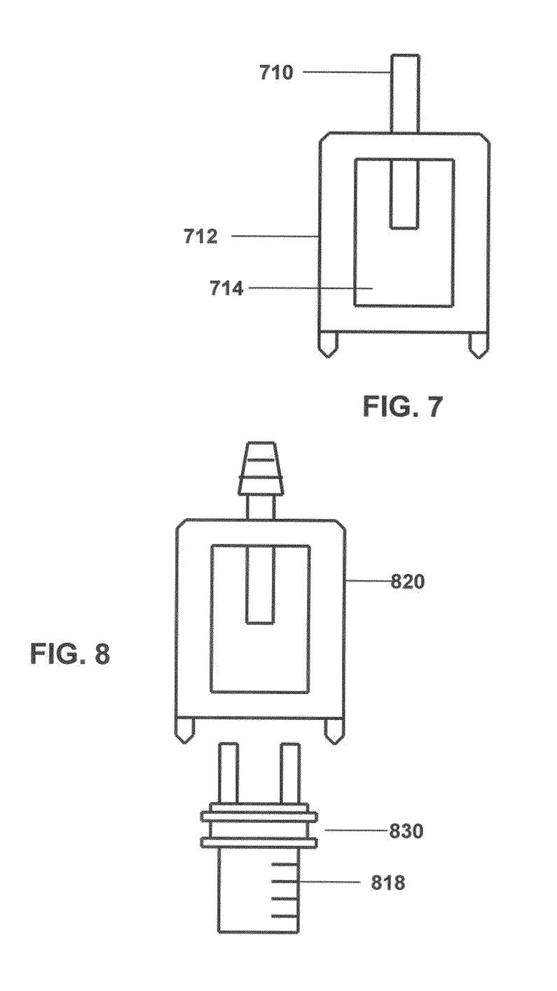

The above description in FIG. 1 is primarily of the vapor mist controller embodiment of the present invention. In the actual construction, the vapor mist controller is fabricated primarily of a durable, medical grade plastic material. The vapor mist control unit of the present invention would be an elongated, tubular apparatus, measuring approximately seventeen inches (17″), two and one half inches (2½″) in width, five inches (5″) in depth, and two to two and one half inches (2″-2½″) in circumference at its base. The top or proximal end of the unit would consist of a capped oxygen connection port 710 configured with an oxygen adjustment multi-vent barrel (controlled device) 712 that comes with interchangeable diluters 830 (see FIGS. 7 and 8) for appropriate oxygen selection to help control and dispense oxygen flow. The diluters can be color coded in green and white to distinguish the settings. Settings for the green diluter can be for percentages of 24%, 26%, 28%, and 30%. Setting for the white diluter can be in percentages of 35%, 40%, and 50%.

Referring to the corrugated tube 130, extending downward, a vertical stem would be comprised of a plastic tee connection outfitted with a horizontal, corrugated tubing valve plug that would connect to a second tee for the unit's nebulizer connection. With an added drainage bag, this connection would extend in line with the corrugated tubing used for collecting the condensing water caused by the humidifier unit. Back on the vertical stem, a one-way flow controlled valve would be provided, and further down, threaded ports would facilitate connection of the vapor mist controller chamber. A bulbous unit, the chamber, would culminate in a handy strainer. The vapor mist controller is compact and lightweight. The entire vapor mist controller could be packaged in a kit form with instructions for easy assembly.

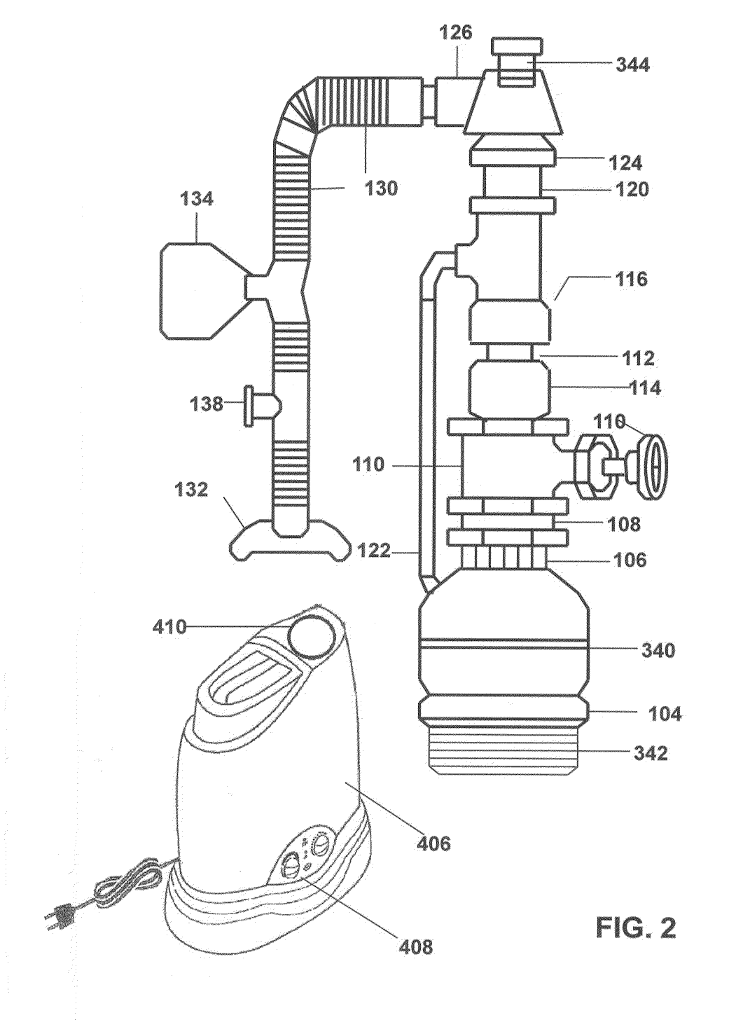

FIG. 2 is an illustration of the system of the present invention incorporating the vapor controller with a standard humidifier device. As shown, the vapor control unit described in FIG. 1 attaches to a humidifier device. The humidifier device comprises the humidifier unit 406. A water tank in the humidifier unit contains distilled water such that the water from tank is used to humidify the air. Controller knobs 408 enable the user to control the amount of moisture and air flow. In the standard humidifier device, a nozzle is attached to an outlet port 410 that emits the moisture and air flow into the atmosphere. In the conventional application of this humidifier unit, the humidifier would just emit the humidified air through the nozzle and into the atmosphere. In the application of the present invention, the humidifier nozzle is removed and the humidifier adapter 104 and strainer 342 of the vapor mist controller insert into the outlet port 410 of the humidifier device. Instead of the humidified vapor mist being emitted into the atmosphere of the patient's room, the humidified air is channeled through the vapor mist controller and corrugated tube directly to the patient. In one embodiment of the present invention, the strainer 342 attached to the bottom chamber of the vapor controller will allow a patient to insert a vapor pad into the strainer to for a vaporizing mist that will unstop blocked nasal passage.

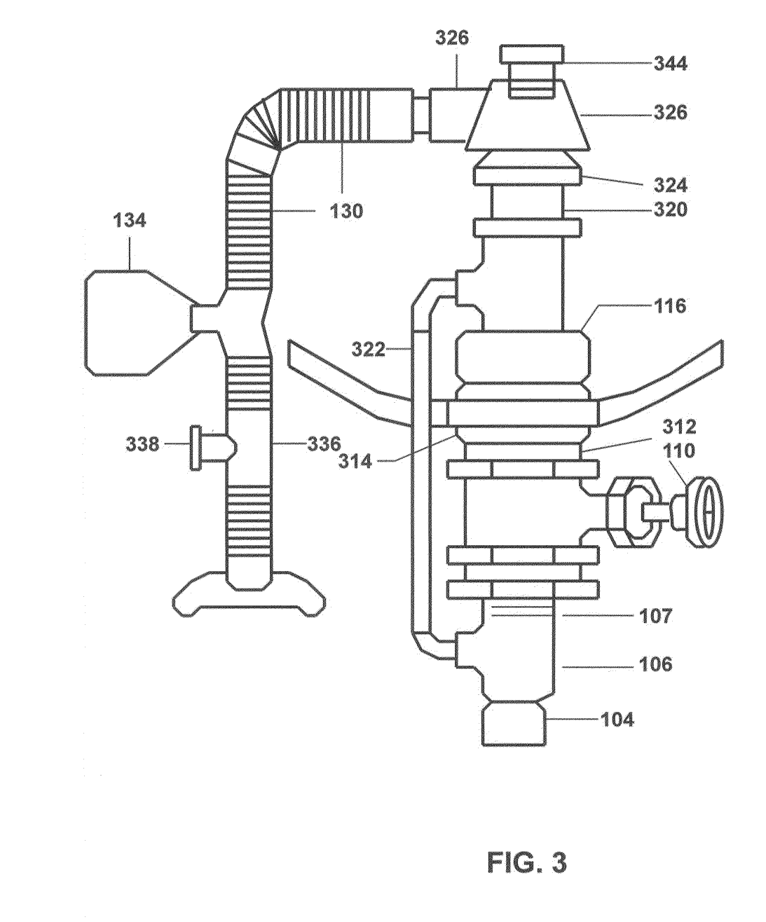

FIG. 3 is an illustration of the mini-vapor controller system of the present invention designed to be attached to a mini-ultrasonic humidifier device. This configuration is similar to the standard vapor mist controller system described in FIG. 1. In this system, a plastic adapter 104 attaches to the bottom of the mini-vapor mist controller. This adapter connects the vapor mist controller to the mini-ultrasonic humidifier device 304. Attached to the adapter 104 is a pressure line adapter 106.

Inserted into the top end of the pressure line adapter 106 is male adapter 107. The bottom end of this male adapter is inserted into the top end of the pressure line adapter 106. The top end of the male adapter can have threads for engaging a flow control valve 110. This control valve 110 is a one-way flow control valve. The valve has a bottom end connected to the male adapter. Humidified vapor enters the valve from the male adapter and flows out of the valve through the upper end. An operator can use the control knob to adjust the flow of humidified vapor through the valve. A PVC pipe nipple 312 connects the flow control valve to a reducing bushing 314. The reducing bushing can be ¾ inch by ½ inch. A pressure line adapter 116 is connected to a second PVC pipe nipple 312 and reducing bushing 314. This pressure line adapter 116 can comprise a plastic/nylon material. The inner diameter is approximately 22 millimeters.

This pressure line adapter 116 also has an opening for insertion of a multi-vent barrel 320. Also connected to the pressure line adapter 116 is a pressure tube 322. The pressure tube 322 forms a loop between the two pressure line adapters 106 and 116. This loop helps regulate the pressure of the humidified vapor mist traveling through the mini-vapor mist controller.

The multi-bent barrel 320 can be an optional component of the vapor mist controller. The multi-vent barrel is used to control the amount of vapor mist released from the vapor mist controller to the patient. A mist retainer and adjustment control locking ring 324 fits around and between the multi-vent barrel and the high humidity adapter 326. The high humidity adapter 326 can have a generally circular shape with a bottom opening and a side opening. The bottom opening connects to the multi-vent barrel and receives the vapor mist flowing though the multi-vent barrel. The side opening is connected to the corrugate tube 130 and channels the vapor mist into the corrugated tube. This configuration of the mini-mist controller has a plug 344. Other configurations of the mini-mist controller can have other features such as the ability to mix oxygen with the vapor mist from the humidifier. Those configurations could have an oxygen diluter instead of a plug. However, in this configuration, there is no mixing of oxygen with the humidified vapor mist. The opening in the top of the high humidity adapter is filled with plug 344. As the vapor mist exits the high humidity adapter 326 it enters the corrugated tubing 130. Elements 134, 336 and 338 are the same as the corresponding elements in FIG. 1.

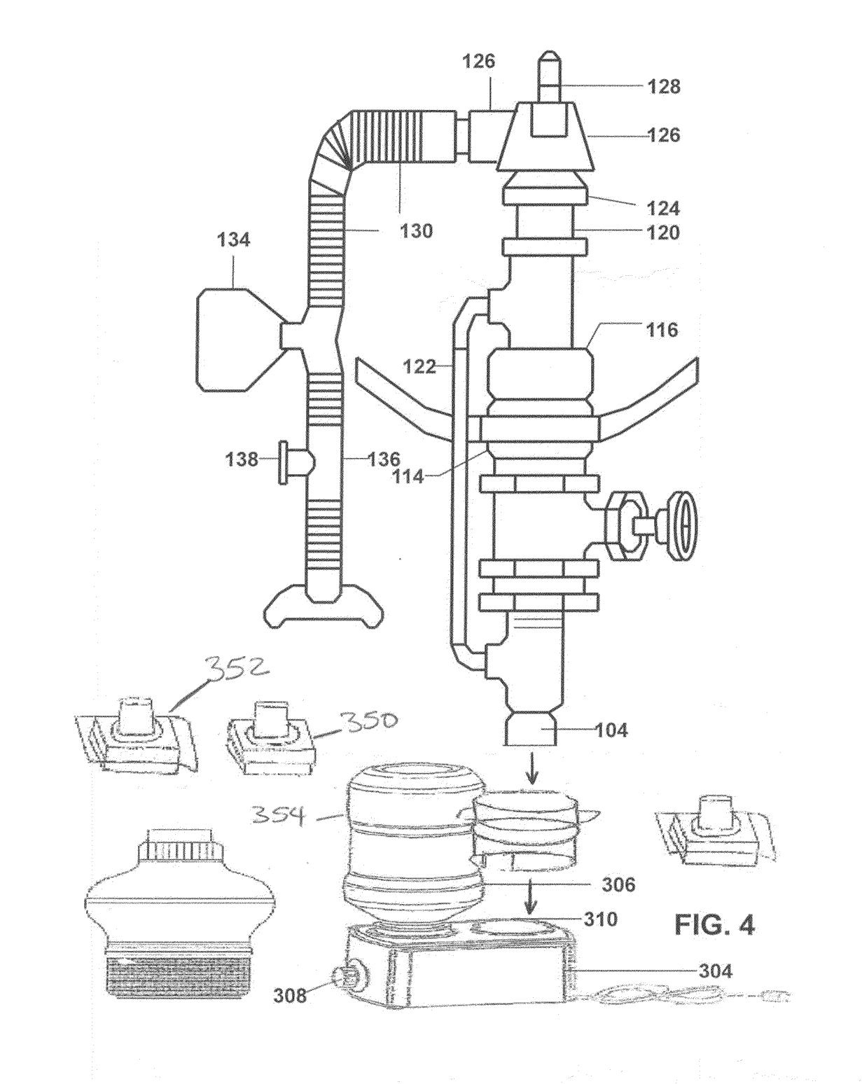

FIG. 4 is an illustration of the system of the present invention incorporating the vapor controller (described in FIG. 3) and a mini-humidifier device. As shown, the vapor control unit attaches to a humidifier device 304. The mini-humidifier device comprises the humidifier unit 304, having a water tank 354 inserted into the mini-humidifier device. The water tank contains distilled water. Controller knobs 308 enable the user to control the amount of humidified air the humidifier unit emits. An outlet port 310 emits the humidified air into the atmosphere. In the conventional application of this humidifier unit, the humidifier would just emit the humidified air into the atmosphere. In the application of the present invention, humidifier adapter 304 of the vapor mist controller inserts into and attaches to the humidifier device in the port outlet 310 via internal adapters such as 350 and 352. An internal adapter can be positioned in the port outlet 310. The humidifier adapter 304 engages the internal adapter 350 to connect the humidifier adapter to the mini-humidifier. Instead of the humidified vapor mist from the mini-humidifier being emitted into the atmosphere of the patient's room, the humidified vapor mist is channeled through vapor mist controller and corrugated tube to the patient.

FIG. 4 is an embodiment of the mini-controller that has the capability to mix oxygen with the humidified vapor mist. Referring to the high humidity adapter 126, in this embodiment, the top opening accommodates an oxygen diluter 128 and multi-vent barrel as described in FIG. 1. As seen, the plug 344 shown in FIG. 3 is replaced by the oxygen diluter 128.

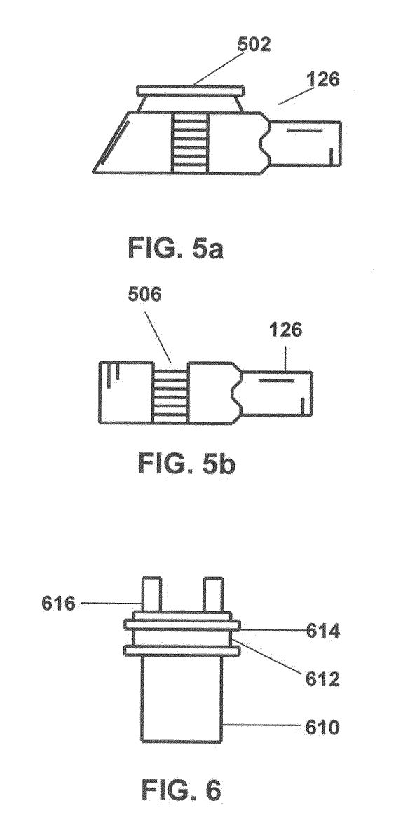

FIGS. 5a and 5b show high humidity adapter embodiments for the vapor controller device. As mentioned, in the present invention, the high humidity adapter 126 can have an opening in its top to receive oxygen port from an oxygen diluter to mix with the vapor mist. If the user does not want to mix oxygen with the vapor mist, as shown in FIGS. 5a, a plug element 502 can be inserted into the high humidity adapter 126. The opening in the top of the high humidity adapter will vary depending on whether an oxygen diluter is attached or a plug. As a result, there can be different high humidity adapters and different vent barrels depending on the particular application. The inlet in the top of the high humidity adapter 126 can be fitted with threads to engage threads fitted at the bottom of both the plug 502 and the oxygen connector. In the alternative, FIG. 5b shows the high humidity adapter without the plug. In this configuration, the oxygen diluter extension 128 can extend through the opening 506 and connect with an oxygen source.

FIG. 6 shows a vent barrel that can be positioned inside the channel of the high humidity adapter 126 in the vapor controller. This vent barrel can have a cylindrical shape with a barrel section 610. Attached to the barrel section is a locking ring 612. The locking ring secures the vent barrel in the humidity adapter. A mist retainer ring 614 serves as a seal to reduce the amount of vapor mist that could escape during the flow of the vapor mist to the high humidity adapter. The top section of the vent barrel is an extension and comprises a cylindrical wall 616 with alternating openings. The openings in the wall help facilitate the flow of vapor mist through and out of the high humidity adapter 126.

FIGS. 7 and 8 show a configuration of an oxygen diluter that can be implemented in the present invention. Similar to the vent barrel, the oxygen diluter has openings to regulate the amount of oxygen that will mix with the vapor mist and flow out of the high humidity adapter. A connector 710 connects the oxygen diluter with an oxygen source. The oxygen diluter has a cylindrical wall 712 with alternating openings 714 in the wall.

FIG. 8 shows the engagement of the oxygen diluter 820 described in FIG. 8 with the vent barrel 830. Markings 818 indicate various settings on the barrel as previously described. The vent barrel openings can be adjusted to various sizes to adjust the amount of vapor mist flowing out of the high humidity adapter and oxygen diluter. As shown, the vent barrel 830 is inserted into the oxygen diluter 820. The locking ring 612 and the high humidity adapter 126 secure the vent barrel and oxygen diluter. As mentioned, the oxygen diluter is an option component that the user will incorporate when there is a desire to supply oxygen along with the vapor mist.

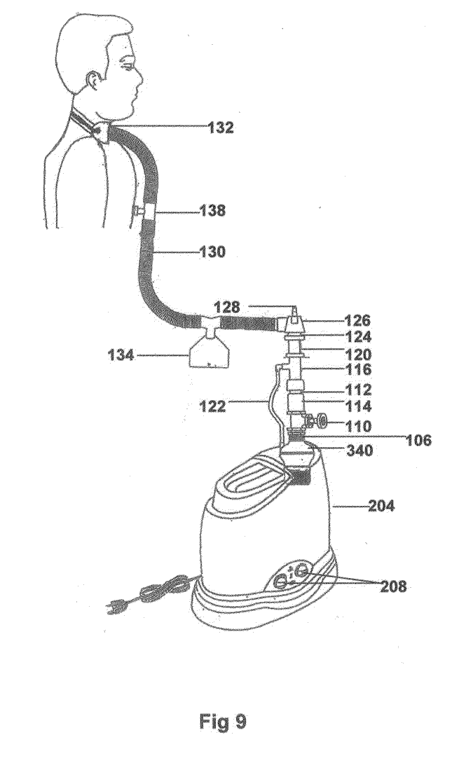

FIG. 9 is an illustration of the implementation of the system of the present invention using a standard ultrasonic humidifier device 204. As shown and previously described, the humidifier device 204 connects to and transmits humidified vapor mist through a controller device. The controller device connects to the corrugated tubing member 130. The vapor mist flows through the corrugated tubing to the patient. As shown, the tubing connects to the patient via a trachea mask 132.

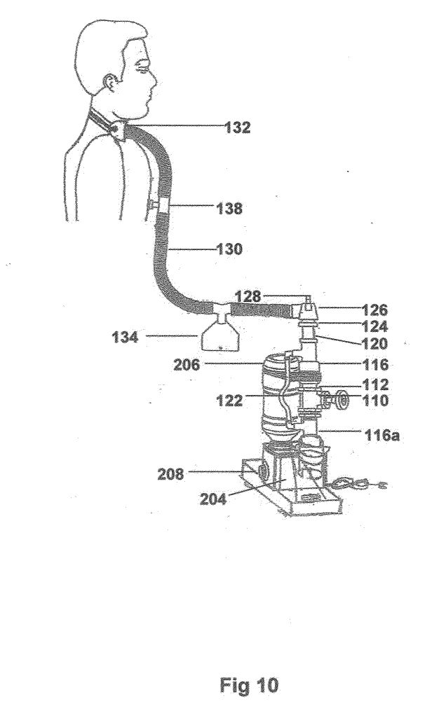

FIG. 10 is an illustration of the implementation of the system of the present invention using a previously described mini-humidifier device 204. Similar to the system configuration in FIG. 9, vapor mist from the humidifier device flows through the controller device attached to the humidifier device. The vapor mist flow continues through the tubing member 130 to the patient.

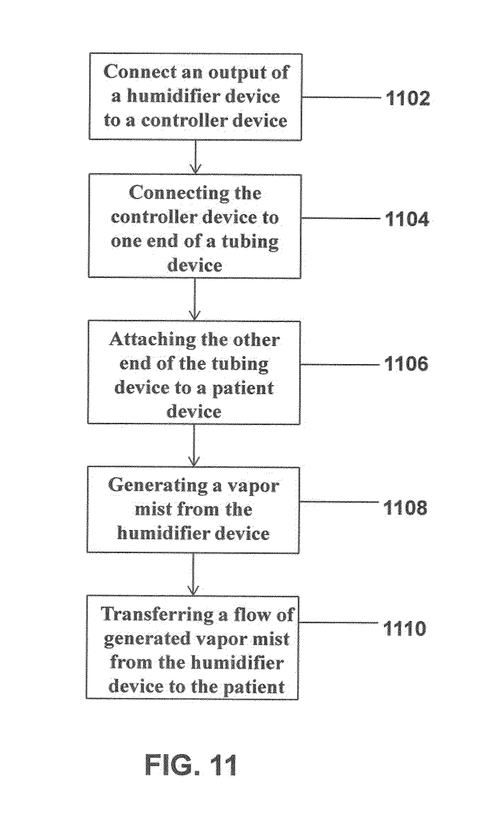

FIG. 11 illustrates the steps in a general implementation of the method of the present invention. In this method, a humidifier device is used to generate a vapor mist that will flow directly to the patient. As mentioned, this approach is more efficient and effective than the conventional method of humidifying a room and having the patient inhale the humidified air in the room. In this method, in step 1102 the output port of the humidifier device is connected to a controller device. Generally, the output nozzle is removed from the humidifier device and the controller attached. In some instances, one or more adapters may be used to facilitate the connecting of the controller device to the humidifier device. Step 1104 connects the controller device to a corrugated tubing member. The vapor mist from the humidifier device will flow through the tubing to the patient and thereby provide a supply of humidified vapor directly to the patient. It should be noted that the tubing member can be directly connected to the humidifier device. The controller device can be optional, but it is preferred to facilitate one's ability to have optimal regulation of the flow of vapor mist to the patient. Step 1106 attaches the other end of the tubing member to the patient device. Depending on the particular treatment the patient is receiving, the patient device can vary and can be for example a trachea mask or an aerosol mask. In step 1108, the humidifier device generates a vapor mist. In step 1110, this generated vapor mist is transferred via the controller device and tubing member directly to the patient.

This invention provides significant advantages over the current art. The invention has been described in connection with its preferred embodiments. However, it is not limited thereto. Changes, variations and modifications to the basic design may be made without departing from the inventive concepts in this invention. In addition, these changes, variations and modifications would be obvious to those skilled in the art having the benefit of the foregoing teachings. All such changes, variations and modifications are intended to be within the scope of this invention.

I claim:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A humidity controller device for generating and controlling a high humidity vapor flow to a patient comprising:
a humidity adapter for engaging and connecting to a humidity vapor generating device, said humidity adapter having two open ends and a channel there though to facilitate a flow of vapor from the humidity vapor generating device into and out of said humidity adapter through the open ends;
a flow control valve connected to said humidity adapter for controlling a flow of vapor from the humidity vapor generating device through said humidity adapter, said flow control valve being a one-way control valve and having two open ends and a channel there through, said flow control valve being attached and aligned with said humidity adapter such that the humidity adapter channel and the flow control valve channel form a continuous channel, said flow control valve having the capability for manual control of vapor flow through said valve; and
a high humidity adapter for generating an increase in humidity in the humidity vapor flowing from the humidity vapor generating device, said high humidity adapter having an inlet opening for engaging and connecting to said flow control valve, said high humidity adapter also having an outlet opening to enable high humidity vapor to a patient, said high humidity adapter having a channel formed between the inlet and outlet openings, said high humidity adapter also capable of increasing humidity in the vapor generated by the humidity vapor generating device resulting in a high humidity vapor that provides more effective relief to a patient.

18. The humidity controller device as described in claim 17 further comprising an oxygen diluter component incorporated into the said high humidity adapter, said oxygen diluter having a cylindrical shaped wall, with a open bottom side forming a channel for receiving humidity vapor from the humidity vapor generating device, said oxygen diluter having and a closed top side with an opening through which an oxygen is inserted to facilitate a flow of oxygen from an oxygen source into said oxygen diluter, said oxygen diluter further having multiple openings of various dimensions in the cylindrical shaped wall for controlling oxygen quantities that are mixed with the humidity vapor.

19. The humidity controller device as described in claim 17 further comprising:
a vapor pressure line adapter positioned between and attached to said high humidity adapter and said flow control valve, said vapor pressure adapter having inlet and outlet openings and an internal channel consist with channels of said flow control valve and high humidity adapter wherein connected components form a continuous channel throughout the humidity adapter and
a pressure line comprising a tube with two open ends, said pressure line being connected to a side opening in side vapor pressure line adapter through one tube end and said pressure line also connected to said humidity adapter through a second pressure line tube end and thereby creating a pressure loop in the humidity controller device for adjusting the pressure of vapor traveling through the humidity controller.

20. The humidity controller device as described in claim 18 further comprising an adjustable control locking ring for securing said oxygen diluter to said high humidity adapter.

21. The humidity controller device as described in claim 17 further comprising a vapor mist retainer element positioned around said locking ring to serve as a seal to restrict vapor inside said high humidity adapter from escaping.

22. The humidity controller device as described in claim 19 wherein said humidity adapter further comprising an adaptive fitting for engaging and attaching said pressure line to said humidity adapter and thereby enabling said humidity adapter to function as a second pressure adapter.

23. The humidity controller device as described in claim 17 wherein said humidity adapter further comprises a detachable strainer component for container a vapor material that will mix with humidity vapor from a humidity vapor generating device as vapor enters said humidity adapter.

24. The humidity controller device as described in claim 19 further comprising a multiple vent barrel having two open ends and a channel through, said multiple vent barrel having one open end attached to said vapor pressure line adapter and the other open end attached to said oxygen diluter and locking ring.

25. A system for generating and transmitting humidity vapor mist to a patient comprising:
a humidity vapor generating device for creating an initial humidity vapor mist;
a humidity vapor mist channel through which humidity vapor flows from said high humidity vapor generating device to a patient, said channel comprising a corrugated tubing and having first end and a second end, said first end being attached to said humidity vapor generating device; and
a patient mask for engaging a patient to facilitate contact of the high humidity vapor with the patient, said mask being attached to the second end of said corrugated tubing.

26. The system for generating and transmitting humidity vapor mist to a patient as described in claim 25 further comprising:
a humidity vapor controller device attached to said humidity vapor mist generating device and attached to said a humidity vapor mist channel and further comprising:
a humidity adapter for engaging and connecting to a humidity vapor generating device, said humidity adapter having two open ends and a channel there though to facilitate a flow of vapor from the humidity vapor generating device into and out of said humidity adapter through the open ends;
a flow control valve connected to said humidity adapter for controlling a flow of vapor from the humidity vapor generating device through said humidity adapter, said flow control valve being a one-way control valve and having two open ends and a channel there through, said flow control valve being attached and aligned with said humidity adapter such that the humidity adapter channel and the flow control valve channel form a continuous channel, said flow control valve having the capability for manual control of vapor flow through said valve; and
a high humidity adapter for generating an increase in humidity in the humidity vapor flowing from the humidity vapor generating device, said high humidity adapter having an inlet opening for engaging and connecting to said flow control valve, said high humidity adapter also having an outlet opening to enable high humidity vapor to a patient, said high humidity adapter having a channel formed between the inlet and outlet openings, said high humidity adapter also capable of increasing humidity in the vapor generated by the humidity vapor generating device resulting in a high humidity vapor that provides more effective relief to a patient.
a drain bag attached to said corrugated tubing for collecting condensation formed from the high humidity vapor flowing through said corrugated tubing; and
a patient mask for engaging a patient to facilitate contact of the high humidity vapor with the patient, said mask being attached to the second end of said corrugated tubing.

27. The system for generating and transmitting high humidity to a patient as described in claim 26 further comprising a nebulizer device attached to said corrugated tubing for facilitating patient breathing, said nebulizer device being attached to said corrugated tubing between said drain bag and said patient mask.

28. The system for generating and transmitting high humidity to a patient as described in claim 26 wherein said patient mask further comprises a trachea mask.

29. The system for generating and transmitting high humidity to a patient as described in claim 26 wherein said patient mask further comprises an aerosol mask.

30. The system for generating and transmitting high humidity to a patient as described in claim 26 further comprising an oxygen connector attached to said a high humidity adapter and an oxygen source for mixing oxygen with humidified vapor to flow through said corrugated tubing.

31. The system for generating and transmitting high humidity to a patient as described in claim 26 further comprising vaporizing material positioned in a detachable strainer to facilitate enhanced breathing of a patient, said vaporizing material and detachable strainer being attached to said humidity adapter of said humidity vapor controller device.

32. A method for generating and transferring a vapor mist created by a humidifier device to a patient comprising:
connecting an output of a humidifier device to one end of a tubing device;
connecting an other end of the tubing device to a patient device;
attaching the patient device to the patient;
generating a vapor mist from a humidifier device; and
transferring a flow of generated vapor mist from the humidifier device through the tubing device to the patient via the patient device.

33. The method for generating and transferring a vapor mist created by a humidifier device to a patient as described in claim 32 wherein said connecting an output of a humidifier device to one end of a tubing device further comprises connecting an output of the humidifier device to a controller device said controller device being connected between the humidifier device and the tubing device and the controller device capable of regulating vapor mist being transferred to the patient.

34. The method for generating and transferring a vapor mist to a patient as described in claim 33 wherein said creating a vapor mist further comprises combining the vapor mist with oxygen, the oxygen being supplied from an external source that is connected to the controller device.

35. The method for generating and transferring a vapor mist to a patient as described in claim 32 further comprising during said transferring of a flow of generated vapor mist, nebulizing the generated vapor mist.

36. The method for generating and transferring a vapor mist to a patient as described in claim 32 wherein said generating a vapor mist from a humidifier device further comprises generating a cool vapor mist and a warm vapor mist.
